(19) United States
(12) Patent Application Publication
(10) Pub. No.: US 2026/0199732 A1
(43) Pub. Date: Jul. 16, 2026

(54) APPARATUS, SYSTEM, AND METHOD FOR RESPIRATORY BIOFEEDBACK

(71) Applicant: UNIVERSITY OF FLORIDA RESEARCH FOUNDATION, INCORPORATED, Gainesville, FL (US)

(72) Inventor: Maribel Z. CIAMPITTI, Jacksonville, FL (US)

(21) Appl. No.: 19/128,012

(22) PCT Filed: Oct. 31, 2023
(86) PCT No.: PCT/US2023/036370

§ 371 Date: May 7, 2025

Related U.S. Application Data
(60) Provisional application No. 63/383,160, filed on Nov. 10, 2022.

(51) Int. Cl. A63B 23/18; A63B 21/00; A63B 24/00; A63B 71/06

(52) U.S. Cl. CPC A63B 23/18; A63B 21/00069; A63B 24/0075; A63B 71/0622; A63B 2024/0068; A63B 2220/56; A63B 2230/40

(57) ABSTRACT

Provided herein is an apparatus, system, and method for measuring respiratory pressures, providing resistance training and respiratory biofeedback. A method described herein for Respiratory Muscle Training (RMT) includes: receiving an indication of initiation of an RMT test using an RMT device; providing a visual indication of a target vent aperture setting; receiving an image of a vent aperture of the RMT device; determining if a setting of the vent aperture in the image is within a predetermined range of the target resistance training range; providing instructions to commence with the RMT training set in response to the setting of the vent aperture in the image being within the predetermined range of the target resistance training pressure level; and providing instructions to reset the vent aperture of the RMT device in response to the setting of the vent aperture in the image not being within the predetermined training target goal.

### CROSS-REFERENCE TO RELATED APPLICATIONS

This application is a national stage entry of PCT Application No PCT/US2023/036370, filed on Oct. 31, 2023, which claims priority to U.S. Provisional Patent Application Ser. No. 63/383,160, filed on Nov. 10, 2023, the contents of which are hereby incorporated by reference in their entirety.

### ACKNOWLEDGEMENT OF FUNDING

This invention was made with government support under Grant No. R41 HL176339, awarded by the National Institutes of Health. The government has certain rights in this invention.

### TECHNOLOGICAL FIELD

The present disclosure relates to respiratory biofeedback, and more particularly, to an apparatus, system, and method for respiratory biofeedback capable of measuring respiratory pressures and providing resistance training to an individual while providing real time feedback.

### BACKGROUND

Respiratory Muscle Training (RMT) is a therapeutic technique that aims to improve function of respiratory muscles when they are weakened from disease or deconditioning. The process for measuring the strength of the respiratory muscles typically involves measuring the maximum inspiratory and expiratory pressures using a manometer device. RMT is accomplished by having a person inhale or exhale against a set “load” or resistance level. Performance can be enhanced by monitoring the quality, consistency, and strength of air flow flowing through a device, the primary purpose of the RMT device is to be able to provide the training load or resistance. Air driven by the lungs flows through an aperture to create a pressure within a device that is measured and monitored. In an RMT device, a pressure differential of either a negative or positive force with respect to atmospheric pressure is created by lung function, through inspiratory or expiratory action, using an aperture. The size of the aperture controls the range of training resistance.

### SUMMARY

An objective of this present disclosure is to provide an apparatus, system, and method for respiratory pressure measurements and biofeedback capable of measuring and training an individual while providing real time feedback. Embodiments provided herein include a method for Respiratory Muscle Training (RMT) including: receiving an indication of initiation of a Respiratory Muscle Strength Test (RMST) using an RMT device; providing a visual indication of a target vent aperture setting; receiving an image of a vent aperture of the RMT device; determining if a setting of the vent aperture in the image is within a predetermined range of the target vent aperture setting; providing instructions to commence with the RMT training in response to the setting of the vent aperture in the image being within the predetermined range of the target vent aperture setting; and providing instructions to reset the vent aperture of the RMT device in response to the setting of the vent aperture in the image not being within the predetermined range of the target vent aperture setting.

According to some embodiments, the indication of initiation of the respiratory strength test includes receiving RMST parameters. According to certain embodiments, the method includes calculating the target vent aperture setting based on the RMST parameters. The method of some embodiments further includes prompting a user to move the vent aperture of the RMT device to the target vent aperture setting. According to some embodiments, determining if a setting of the vent aperture in the image is within a predetermined range of the target vent aperture setting includes: calculating open areas of the vent aperture relative to closed areas based on the image of the vent aperture of the RMT device; and determining the predetermined range of the target vent aperture setting based on an ability to conduct the RMST with the calculated open areas of the vent aperture in relation to flow rate. The visual indication of the target vent aperture setting includes, in some embodiments, an image of a vent aperture of a reference RMT device set to the target vent aperture setting. The method of some embodiments includes receiving, from the RMT device, pressure data from the RMST. The method of some embodiments further includes processing the pressure data based on the setting of the vent aperture to establish results of the RMST and providing a visual indication of the results of the RMST.

Embodiments of the present disclosure include a Respiratory Muscle Training (RMT) system including: an RMT device; and a user device including a user interface, where the user device is configured to: receive an indication of initiation of an RMST using the RMT device; provide a visual indication of a target vent aperture setting; receive an image of a vent aperture of the RMT device; determine if a setting of the vent aperture in the image is within a predetermined range of the target vent aperture setting; provide instructions to commence with the RMST in response to the setting of the vent aperture in the image being within the predetermined range of the target vent aperture setting; and provide instructions to reset the vent aperture of the RMT device in response to the setting of the vent aperture in the image not being within the predetermined range of the target vent aperture setting.

According to some embodiments, the user device configured to receive the indication of initiation of the RMST includes the user device configured to receive RMST parameters from a user via the interface. The user device of an example embodiment is further configured to calculate the target vent aperture setting based on the RMST parameters. The user device of an example embodiment is further configured to prompt a user to move the vent aperture of the RMT device to the target vent aperture setting. According to some embodiments, the user device configured to determine if a setting of the vent aperture in the image is within a predetermined range of the target vent aperture setting includes the user device configured to: calculate open areas of the vent aperture relative to closed areas based on the image of the vent aperture of the RMT device; and determine the predetermined range of the target vent aperture setting based on an ability to conduct the RMST with the calculated open areas of the vent aperture. The visual indication of the target vent aperture setting includes, in some embodiments, an image of a vent aperture of a reference RMT device set to the target vent aperture setting presented on the user interface. The user device of an example embodiment is further configured to receive, from the RMT device, pressure data from the RMST. The user device of an example embodiment is further configured to: process the pressure data based on the setting of the vent aperture to establish results of the RMST; and provide a visual indication of the results of the RMST via the user interface.

Embodiments provided herein include a Respiratory Muscle Training (RMT) device including: a body defining a chamber; a pressure exchange area, where the pressure exchange area is separated from the chamber by a diaphragm; an electronics section removably attached to the body, where a pressure sensor of the electronics section detects a pressure within the pressure exchange area; and a vent, where the vent enables air received into the chamber from a mouthpiece to exit the chamber. The pressure exchange area of an example embodiment is sealed to the atmosphere when the electronics section is removed from the body. The electronics section of an example embodiment includes a communication interface, where the communication interface is configured to transmit data from the pressure sensor to a user device. The RMT device of an example embodiment includes an orifice defined in the vent, where the orifice functions as a bleed hole for the chamber.

### BRIEF DESCRIPTION OF THE DRAWINGS

Some embodiments of this present disclosure may be further understood by the detailed descriptions and corresponding figures.

FIG. 1 illustrates a simplified version of an RMT device without a mouthpiece according to an example embodiment of the present disclosure;

FIG. 2 illustrates a section view of an RMT device including the mouthpiece according to an example embodiment of the present disclosure;

FIG. 3 illustrates a body of the RMT device separated from the electronics section of the RMT device according to an example embodiment of the present disclosure;

FIG. 4 illustrates a vent of an RMT device in three different stages of opening of the aperture according to an example embodiment of the present disclosure;

FIG. 5 illustrates a user device having a user interface for use with a software application according to an example embodiment of the present disclosure;

FIG. 6 illustrates a block diagram of an apparatus that can be employed as the user device or the electronics section of the RMT device according to an example embodiment of the present disclosure;

FIG. 7 illustrates a flow chart of operations of an RMST using the RMT device and software application according to an example embodiment of the present disclosure;

FIG. 8 illustrates a vent of an RMT device including an orifice to improve accuracy according to an example embodiment of the present disclosure; and

FIG. 9 is a flowchart of a method of operating an RMT system according to an example embodiment of the present disclosure.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements.

Embodiments of the present disclosure provide an apparatus, system, and method for respiratory biofeedback capable of measuring and training an individual while providing real time feedback. Respiratory Muscle Training (RMT), as noted above, is a therapeutic technique that is intended to improve function of respiratory muscles, such as those weakened from disease or deconditioning. While RMT is often used for individuals with compromised breathing abilities, RMT has also been employed in athletic training to optimize lung function. RMT can improve performance in athletes, increase endurance for vocal performers and wind-instrument players, and benefit professional public speakers.

Weakness of breathing muscles an adversely affect an individual's ability to be physically active, and thus can have long-term negative consequences. Improvement of breathing ability is important to overall health, and particularly to improve the health and well-being of individuals with compromised breathing function, such as individuals who have asthma, emphysema, neurodegenerative disease, trauma, spinal cord injuries, COPD, or who have had a stroke.

Embodiments provided herein include a system for RMT that promotes patient adherence through ease of operation and rapid benefit realization, in addition to a method of operation that tracks performance and can be remotely monitored to identify improvements or regression. The device of example embodiments can be used for measurement and tracking of both maximal inspiratory pressure (MIP) and maximal expiratory pressure (MEP) without requiring separate devices.

Mechanical RMT devices use a mechanical spring and diaphragm where the force created by lung function presses against the spring which provides resistance. Pressure created by the lung force for the purposes of RMT must overcome the spring force in a vessel to allow high pressure from the lungs to vent to atmospheric pressure. Without sufficient force, the spring will not permit air flow to occur around the diaphragm, such that the lung force has to increase until the spring force is exceeded, and the stopper is displaced. Once the lung force has overcome the spring force, the lung force must be maintained with sufficient air flow to keep the spring open. Existing mechanical devices are unable to capture and save performance data to track RMT progress over time, and in some cases, require separate devices to measure MIP and MEP.

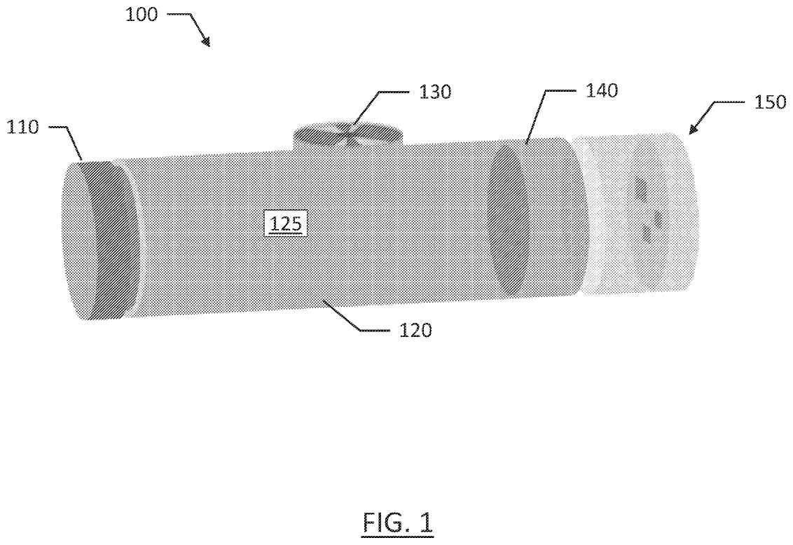

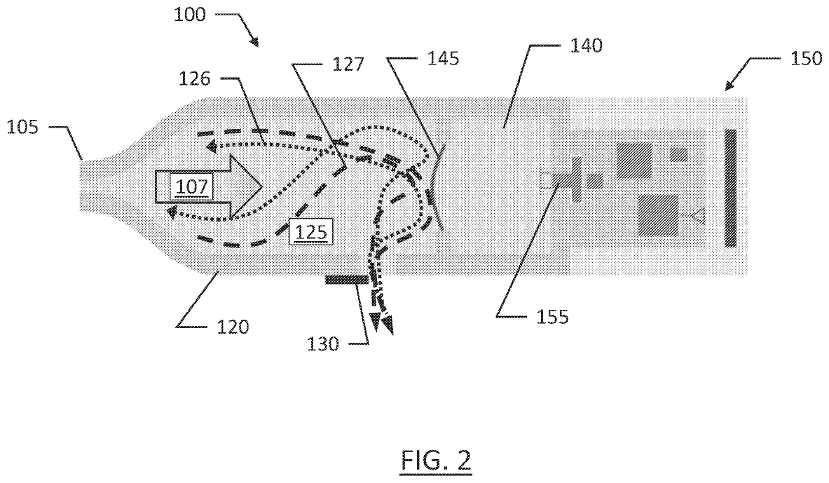

Embodiments described herein provide an electronic RMT device that is capable of measuring MIP and MEP and providing biofeedback during exercise training while also being able to record historic performance to track progress over time. FIG. 1 illustrates a simplified example embodiment of an RMT device 100 of the present disclosure without a mouthpiece. As shown, the RMT device 100 includes a mouthpiece area 110, a body 120 defining a chamber, a vent 130 between the chamber and the atmosphere, pressure exchange area 140, and electronics section 150. FIG. 2 illustrates a section view of an RMT device 100 including the mouthpiece 105 and air flow along arrow 107, where the air travels through the chamber 125 represented by dashed-line arrows 127 and exits through the vent 130. The pressure within the chamber 125 presses against diaphragm 145 to communicate the pressure within the chamber 125 to the pressure exchange area 140. The pressure within the pressure exchange area 140 is sensed by pressure sensor 155 which is in communication with the electronics section 150. Air can also be inhaled through the RMT device 100 as depicted by dotted-line arrows 126 during inhalation in a direction opposite the direction of arrow 107. During inhalation, pressure within pressure exchange area presses against diaphragm 146 due to a lower pressure in chamber 125.

The housing of the electronics section 150 can be, in an example embodiment, removable from the body 120. The body may be a replaceable component as it is designed to not include relatively expensive elements of the RMT device 100 as found in the electronics section 150. While the body 120 may be disposable, the body may optionally be washable, such that the body can be re-used either by the same user. The simplicity of the body 120 renders the cleaning thereof relatively easy. The pressure exchange area 140 is sealed from the chamber 125 by the diaphragm 145, which precludes the breath of a user from directly contacting the pressure sensor 155, thereby preserving the pressure sensor's sterility. The pressure exchange area 140 transfers the pressure from the chamber 125 to the pressure sensor 155 without exposing the electronics section 150 to air from the user's lungs. This precludes moisture, bacteria, and viral load from interfacing with the pressure sensor 155 thereby keeping the electronics section 150 sterile. The electronics section 150 could, after use of the RMT device 100, be transferred from the body 120 to a second body 120 with minimal cleaning necessary to fully disinfect the electronics section 150 without risking cross-contamination between users.

The diaphragm 145 can be constructed of a material that has a relatively uniform material durometer to maintain a spring constant variation of the component low such that differences across bodies such as body 120 are minimal. This can avoid any calibration of the pressure sensor 155 or other electronics. The material can be manufactured in a variety of possible ways, including but not limited to, being die-cut medical grade rubber, for example, or can be poured in place using a one-part molded rubber or a mold and a two-part flowable room temperature vulcanization (RTV) material such as an FDA approved material. With a molded approach, a flowable single part or two-part RTV with the assistance of non-silicone primers could adhere to the plastic of the body 120 as part of the manufacturing process, where diaphragm thickness and therefore spring factor/durometer can be tightly controlled.

The electronics section 150 housing is connected to the body 120 via a quick connect mechanism which can include a twist-to-lock connection, a snap connection, a magnetic connection, or similar quick-connect self-sealing and self-closing on disconnect apparatus. The electronics section 150 must be fastened to the body 120 in such a way as to ensure that the pressure exchange area 140 is sealed air-tight otherwise the pressure is not properly transferred through the pressure exchange area.

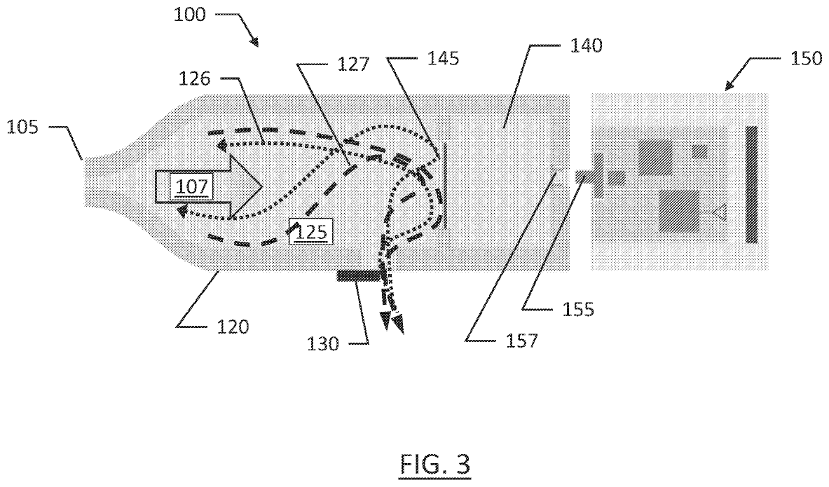

According to some embodiments, the pressure exchange area 140 includes a quick-connect fitting 157 as shown in FIG. 3, with the electronics section 150 separated from the body 120. The quick-connect fitting 157 can be configured to receive the pressure sensor 155 and to transfer pressure from the pressure exchange area 140 to the pressure sensor. The quick-connect fitting 157 of an example embodiment is configured to seal the pressure exchange area 140 when the electronics section 150 is separated from the body 120.

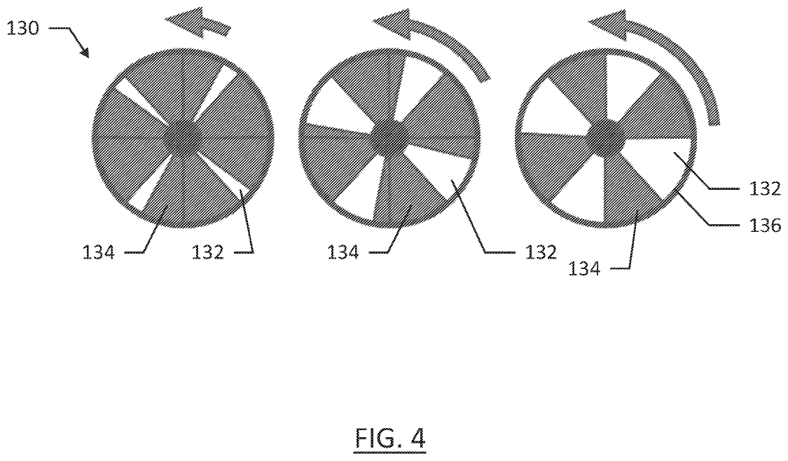

The vent 130 is illustrated separately in FIG. 4 in three stages of opening. As shown, the vent 130 can be rotated to create more open space 132 permitting more air through, while vent vanes 134 occlude the open area of the vent and reduce air flow. While four vanes are shown, any number of vent vanes are possible. The potential open space 132 is limited to about 50% of the total area of the vent 130 with static vent vanes 134 that are fixed to the vent hub and vent periphery 136. However, vent vanes can be stackable thereby allowing more than 50% of the total area of the vent 130 to be available to be opened to air flow. Regardless, the vent 130 can be sized such that the proper amount of air flow through the vent is available depending upon the type of vent and vent vanes used. Further, different types of flow restrictors can be implemented beyond the vent 130 depicted in FIG. 4. For instance, a valve-type flow restriction can be used in some embodiments.

The vent 130 is designed to control a volume of air flow through the chamber 125 depending upon the therapeutic needs. The vent setting of an example embodiment can be determined by a software application associated with the RMT device. The software application of a digital device can be in communication with the electronics section 150, such as through a communications interface described further below. A user may be prompted by a user interface of the software application to set the correct air flow setting of the device using the adjustable vent. With the correct vent setting, pressure can be measured at the pressure exchange area 140 where the air flow can be algorithmically determined knowing the open cross-sectional area of the vent itself.

The vent setting and operation is critical to proper functionality of the RMT device described herein. Thus, the RMT system employs the device 100 depicted in FIGS. 2 and 3 for use with software to accurately record and communicate RMT progress. Further, the software is capable of instructing a user as to the appropriate vent position for optimal performance for the desired function. The software application of example embodiments ensures the correct vent aperture setting with digital feedback. The vent setting is determined based on a target resistance training range for air flowing through the RMT device. The vent set at a target vent setting provides a resistance training pressure level for the RMT training that is selected by a user, a doctor, or a rehabilitation therapist recommended training regimen.

According to an example embodiment, a user selects a desired airflow and pressure combination within a user interface of the software application. The software application, via the user interface, provides a graphical indication of a setting for the vent to be manually adjusted by the user. The user is prompted to make the corresponding changes to the physical vent on the device. Upon the user setting the vent as instructed, the software application prompts a picture of the vent to be taken to validate the user settings. As the vent setting is a manual process, it is subject to human error. The vent setting may be imperfect relative to the instructed vent setting of the software application. With the picture of the vent taken, the software application can determine the precise vent setting. If the vent setting is within a predefined degree of error of the commanded vent setting, the software application can make any necessary adjustments to the software application settings to compensate for the allowable error. The resistance training pressure offered by the vent setting within the predefined degree of error is determined to be sufficient to achieve the goals of the RMT training. If the vent setting is outside of the predefined degree of error, the vent setting may be too far away from the commanded vent setting for compensation to be made within the software, at which point the vent setting by the user is rejected and a re-adjust is requested via the user interface.

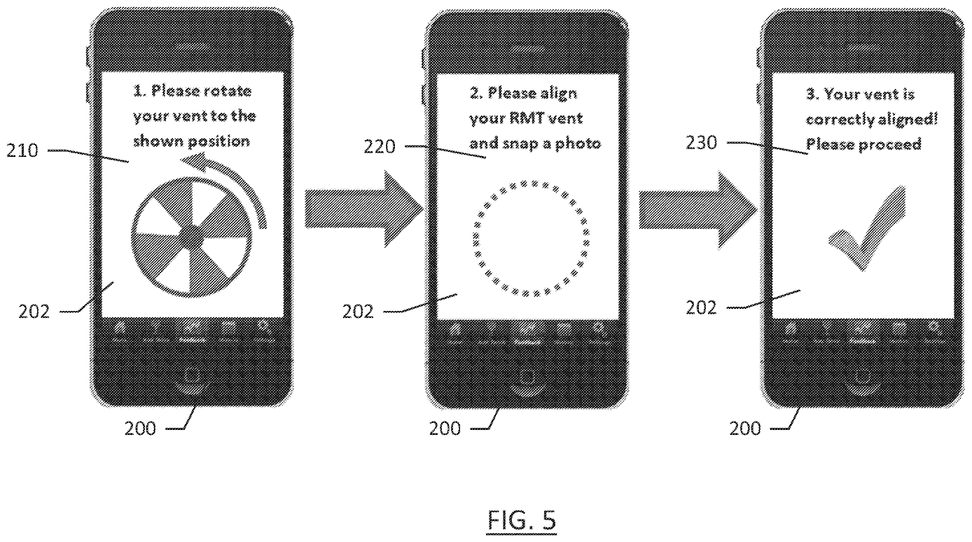

FIG. 5 illustrates an example user device 200 that provides a user interface 202 through which instructions are provided to the user. At 210, the user interface 202 displays an instruction for the user to open the vent 130 to an illustrated configuration. At 220, the user interface 202 instructs a picture to be taken of the vent. At 230, an indication is provided on the user interface 202 that the vent is properly aligned, and the RMT device use may continue.

The precision calculation of the graphical vent aperture from area derived from the picture obtained through the software application allows the software application to more accurately measure air flow and total volume displaced as a function of measured pressure. Embodiments of the user device 200 described herein can be a smart phone, for example, thereby using a user device that is ubiquitous and owned by most adults. This further reduces the expense for a user to use the RMT system described herein. Further, the use of such a user device 200 improves the precision with which data is captured.

The user device 200 can also be in communication with the electronics section 150 of the RMT device 100 to provide enhanced functionality of the RMT device using the software application. The electronics section 150 can include an array of components, but requires at least a power source which can, in some embodiments, be a battery. The battery can be a replaceable battery. Alternatively, the battery can be rechargeable, and can be configured to be wirelessly recharged such as through a charging pad using conventional wireless charging standards. The electronic section further includes a communications interface to communicate with the user device 200. The communications interface can be a wireless communications interface such as a Near-Field Communication (NFC) protocol such as Bluetooth™, or possibly a wired communication interface in some embodiments.

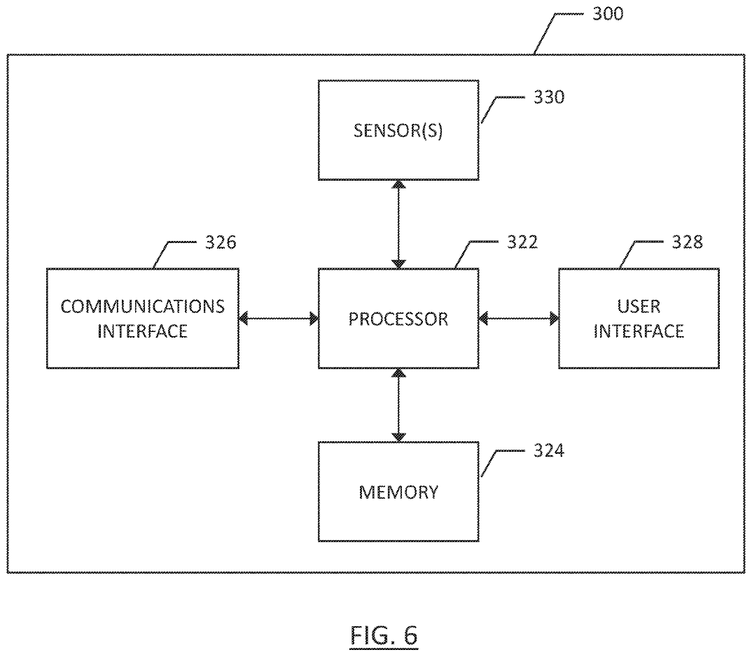

A schematic diagram of an example embodiment of an apparatus 300 that can embody the user device 200 or the electronics section 150 is shown in FIG. 6. As shown, the apparatus 300 may be embodied in a number of different ways, particularly with respect to the electronics section 150. The example apparatus 300 of FIG. 6 includes or is otherwise be in communication with a processor 322, a memory 324, a communication interface 326 and a user interface 328. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

In some embodiments, the processor 322 (and/or co-processors or any other processing circuitry assisting or otherwise associated with the processor) may be in communication with the memory 324 via a bus for passing information among components of the apparatus. The memory 324 may include, for example, one or more volatile and/or non-volatile memories. In other words, for example, the memory 324 may be an electronic storage device (e.g., a computer readable storage medium) comprising gates configured to store data (e.g., bits) that may be retrievable by a machine (e.g., a computing device like the processor).

The memory 324 may be configured to store information, data, content, applications, instructions, or the like for enabling the apparatus 300 to carry out various functions in accordance with an example embodiment of the present disclosure. For example, the memory 324 could be configured to buffer input data for processing by the processor 322. Additionally or alternatively, the memory could be configured to store instructions for execution by the processor.

The processor 322 may be embodied in a number of different ways. For example, the processor 322 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor 322 may include one or more processors configured in tandem via the bus to enable independent execution of instructions, pipelining and/or multithreading. The processor may be embodied as a microcontroller having custom bootloader protection for the firmware from malicious modification in addition to allowing for potential firmware updates.

In an example embodiment, the processor 322 may be configured to execute instructions stored in the memory 324 or otherwise accessible to the processor 322. Alternatively or additionally, the processor 322 may be configured to execute hard coded and/or soft coded functionality. As such, whether configured by hardware or software methods, or by a combination thereof, the processor 322 may represent an entity (e.g., physically embodied in circuitry) capable of performing operations according to an embodiment of the present disclosure while configured accordingly. Thus, for example, when the processor 322 is embodied as an ASIC, FPGA or the like, the processor 322 may be specifically configured hardware for conducting the operations described herein. Alternatively, as another example, when the processor 322 is embodied as an executor of software instructions, the instructions may specifically configure the processor 322 to perform the algorithms and/or operations described herein when the instructions are executed. However, in some cases, the processor 322 may be a processor of a specific device (e.g., the RMT device) configured to employ an embodiment of the present disclosure by further configuration of the processor 322 by instructions for performing the algorithms and/or operations described herein. The processor 322 may include, among other things, a clock, an arithmetic logic unit (ALU) and logic gates configured to support operation of the processor 322. In one embodiment, the processor 322 may also include user interface circuitry configured to control at least some functions of one or more elements of the user interface 328.

Meanwhile, the communication interface 326 may include various components, such as a device or circuitry embodied in either hardware or a combination of hardware and software that is configured to receive and/or transmit data from the apparatus 300 to a network, a server, or a particular user device operating the software application, for example. In this regard, the communication interface 326 may include, for example, an antenna (or multiple antennas) and supporting hardware and/or software for enabling communications wirelessly. Additionally or alternatively, the communication interface 326 may include the circuitry for interacting with the antenna(s) to cause transmission of signals via the antenna(s) or to handle receipt of signals received via the antenna(s). For example, the communication interface 326 may be configured to communicate wirelessly with a head-mounted display, such as via Wi-Fi (e.g., vehicular Wi-Fi standard 802.11p), Bluetooth™, mobile communications standards (e.g., 3G, 4G, or 5G) or other wireless communications techniques. In some instances, the communication interface 326 may alternatively or also support wired communication, which may communicate with a separate transmitting device (not shown). As such, for example, the communication interface 326 may include a communication modem and/or other hardware/software for supporting communication via cable, digital subscriber line (DSL), universal serial bus (USB) or other mechanisms. For example, the communication interface 326 may be configured to communicate via wired communication with other components of a computing device.

The user interface 328 may be in communication with the processor 322, such as the user interface circuitry, to receive an indication of a user input and/or to provide an audible, visual, mechanical, or other output to a user. As such, the user interface 328 may include, for example, one or more buttons, light-emitting diodes (LEDs), a display, a speaker, and/or other input/output mechanisms. The user interface 328 may also be in communication with the memory 324 and/or the communication interface 326, such as via a bus.

The communication interface 326 may facilitate communication between the apparatus 300 and various other devices, networks, or servers. The communication interface 326 may be capable of operating in accordance with various communication protocols, such as licensed and/or non-licensed F.C.C. (Federal Communications Commission, U.S.) protocols or other internationally approved wireless analog and digital communication methods and the like.

The apparatus 300 may optionally include one or more sensors 330, such as a pressure sensor (e.g., pressure sensor 155 of FIG. 2) when the apparatus 300 embodies the RMT device 100.

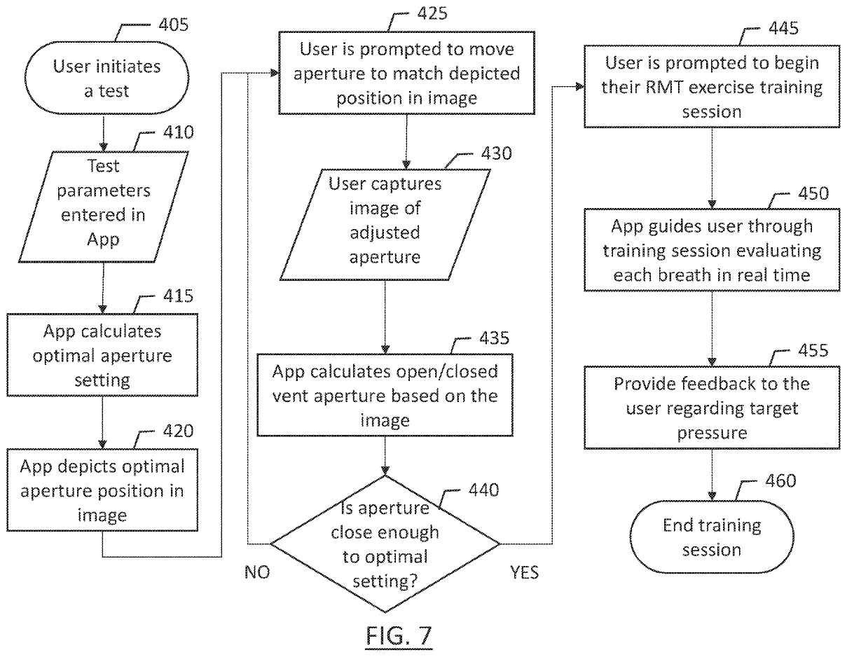

FIG. 7 illustrates an example use case for the RMT system described herein with the RMT device operating together with the software application to measure air flow and generate pressure and volume calculations. By using the calculated area of the aperture, the RMT system can understand the exact area of the aperture as a key variable in air flow without requiring a stepper motor or other mechanical elements to control the vent.

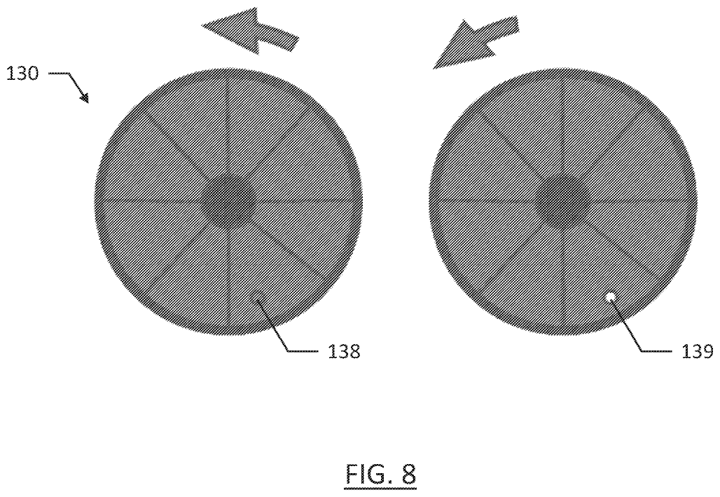

As shown, a user initiates a Respiratory Muscle Strength Test (RMST) on a software application on their user device at 405 to measure either their maximum inspiratory or expiratory pressure (MIP or MEP). Separate manometry devices are not required by example embodiments described herein. Initially, the vent aperture will be set to closed other than the orifice described below with reference to FIG. 8. This test will yield the MIP or MEP in centimeters of water (cm-H2O). The MIP or MEP value generated from the initiated test is the basis for a resistance training level to which the test parameters will be set to, where a user identifies a value that is a percentage (e.g., 50%, 70%, or 80%) of the MIP or MEP value. The user can then enter test parameters at 410 in the app. Optionally, a predefined set of test parameters may be pre-filled based on user preferences or a schedule of tests with predefined parameters. The software application calculates the optimal aperture setting for the vent at 415. The software application can then provide a visual indication of the optimal vent aperture position at 420. The user is prompted at 425 to manually move the vent aperture according to the position depicted on the user device. At 430, the user takes a picture of the vent of the RMT device to depict the open aperture. The user device may prompt this picture capture and may open the camera feature automatically as part of the RMST. The software application at 435 calculates the open/closed areas of the vent in the event. At 440, if the position of the vent is not sufficiently close to the test parameters, the process returns to 425 where the user is prompted to move the aperture to match a depicted vent. If the position of the vent is sufficiently close to the test parameters, the user is prompted at to continue to initiate RMT exercises. The exercises commence at 445 using the photo-measured open aperture. The exercise training involves the user breathing in (inspiratory) or out (expiratory) depending upon the training plan and guided by the app at 450 through the training session, with the RMT device measuring the pressure through pressure sensor 155. Feedback regarding the measured pressure at the pressure sensor 155 relative to a target pressure is provided at 455, such as via the user interface in real-time to the user. The user completes the RMT exercise training session on the software application at 460.

Users, during an RMST, can either wittingly or inadvertently affect desired test pressure and perceived airflow through pressure applied by cheek muscles. Embodiments of the RMT device provided herein include an orifice to mitigate this adverse effect. FIG. 8 illustrates a vent 130 with an orifice, shown closed at orifice 138, and open at 139. This orifice acts as a bleed hole or leak hole and will not allow pressure to accumulate with simple cheek pressure as opposed to full lung pressure. Therefore embodiments of the described RMT device provide more reliable results. The orifice can be, for example, about one millimeter in diameter.

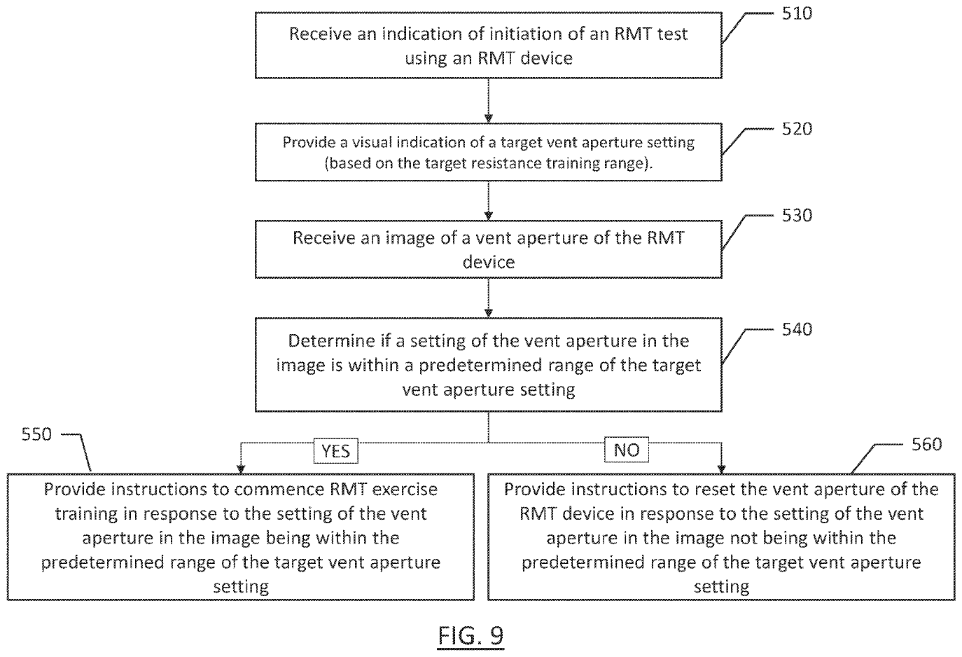

FIG. 9 illustrates operations for a method of Respiratory Muscle Training using the device and software application described herein. As shown in FIG. 9, an indication of initiation of an RMST using an RMT device is received at 510. A visual indication of a target vent aperture setting is provided at 520. An image of a vent aperture of the RMT device is received at 530. From the image, it is determined at 540 if a setting of the vent aperture in the image is within a predetermined range of the target vent aperture setting. In response to the setting of the vent aperture in the image being within the predetermined range of the target vent aperture setting, instructions are provided at 550 to commence with the RMT exercises. In response to the setting of the vent aperture in the image not being within the predetermined range of the target vent aperture setting, instructions are provided at 560 to reset the vent aperture of the RMT device. This may lead back to element 520 where a visual indication of a target vent aperture setting is provided.

FIGS. 7 and 9 illustrate flowcharts of operations configured to facilitate RMST testing. Blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

In an example embodiment, an apparatus for performing the method of FIGS. 7 and/or 9 above may comprise a processor (e.g., the processor 22) configured to perform some or each of the operations (405-450 and/or 510-560) described above. The processor may, for example, be configured to perform the operations (405-450 and/or 510-560) by performing hardware implemented logical functions, executing stored instructions, or executing algorithms for performing each of the operations. Alternatively, the apparatus may comprise means for performing each of the operations described above. In this regard, according to an example embodiment, examples of means for performing operations 405-450 and/or 510-560 may comprise, for example, the processor 22 and/or a device or circuit for executing instructions or executing an algorithm for processing information as described above.

In some embodiments, certain ones of the operations above may be modified or further amplified. Furthermore, in some embodiments, additional optional operations may be included. Modifications, additions, or amplifications to the operations above may be performed in any order and in any combination.

Any modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A method for Respiratory Muscle Training (RMT) comprising:
receiving an indication of initiation of Respiratory Muscle Strength Test (RMST) using an Respiratory Muscle Training (RMT) device;
providing a visual indication of a target vent aperture setting;
receiving an image of a vent aperture of the RMT device;
determining if a setting of the vent aperture in the image is within a predetermined range of the target vent aperture setting;
providing instructions to commence with the RMST in response to the setting of the vent aperture in the image being within the predetermined range of the target vent aperture setting; and
providing instructions to reset the vent aperture of the RMT device in response to the setting of the vent aperture in the image not being within the predetermined range of the target vent aperture setting.

2. The method of claim 1, wherein receiving the indication of initiation of the RMST comprises receiving RMST parameters.

3. The method of claim 2, further comprising:
calculating the target vent aperture setting based on the RMST parameters and selected target training range.

4. The method of claim 1, further comprising:
prompting a user to move the vent aperture of the RMT device to the target vent aperture setting.

5. The method of claim 1, wherein determining if a setting of the vent aperture in the image is within a predetermined range of the target vent aperture setting comprises:
calculating open areas of the vent aperture relative to closed areas, relative to flow rates, based on the image of the vent aperture of the RMT device; and
determining the predetermined range of the target vent aperture setting based on an ability to conduct the RMST with the calculated open areas of the vent aperture.

6. The method of claim 1, wherein the visual indication of the target vent aperture setting comprises an image of a vent aperture of a reference RMT device set to the target vent aperture setting.

7. The method of claim 1, further comprising:
receiving, from the RMT device, pressure data from the RMST.

8. The method of claim 7, further comprising:
processing the pressure data based on the setting of the vent aperture to establish results of the RMST; and
providing a visual indication of the results of the RMST and training exercises.

9. A Respiratory Muscle Training (RMT) system comprising:
a Respiratory Muscle Training (RMT) device; and
a user device comprising a user interface, wherein the user device is configured to:
receive an indication of initiation of a Respiratory Muscle Strength Test (RMST) using the RMT device;
provide a visual indication of a target vent aperture setting;
receive an image of a vent aperture of the RMT device;
determine if a setting of the vent aperture in the image is within a predetermined range of the target vent aperture setting;
provide instructions to commence with the RMST in response to the setting of the vent aperture in the image being within the predetermined range of the target vent aperture setting; and
provide instructions to reset the vent aperture of the RMT device in response to the setting of the vent aperture in the image not being within the predetermined range of the target vent aperture setting.

10. The system of claim 9, wherein the user device configured to receive the indication of initiation of the RMST comprises the user device configured to receive RMST parameters from a user via the user interface.

11. The system of claim 10, wherein the user device is further configured to:
calculate the target vent aperture setting based on the RMST parameters.

12. The system of claim 9, wherein the user device is further configured to:
prompt a user to move the vent aperture of the RMT device to the target vent aperture setting.

13. The system of claim 9, wherein the user device configured to determine if a setting of the vent aperture in the image is within a predetermined range of the target vent aperture setting comprises the user device configured to:
calculate open areas of the vent aperture relative to closed areas based on the image of the vent aperture of the RMT device; and
determine the predetermined range of the target vent aperture setting based on an ability to conduct the RMST with the calculated open areas of the vent aperture.

14. The system of claim 9, wherein the visual indication of the target vent aperture setting comprises an image of a vent aperture of a reference RMT device set to the target vent aperture setting presented on the user interface.

15. The system of claim 9, wherein the user device is further configured to:
receive, from the RMT device, pressure data from the RMT device;

16. The system of claim 15, wherein the user device is further configured to:
process the pressure data based on the setting of the vent aperture to establish results of the RMST; and
provide a visual indication of the results of the RMST via the user interface.

17. A Respiratory Muscle Training (RMT) device comprising:
a body defining a chamber;
a pressure exchange area, wherein the pressure exchange area is separated from the chamber by a diaphragm;
an electronics section removably attached to the body, wherein a pressure sensor of the electronics section detects a pressure within the pressure exchange area; and
a vent, wherein the vent enables air received into the chamber from a mouthpiece to enter or exit the chamber.

18. (canceled)

19. The RMT device of claim 17, wherein the electronics section comprises a communication interface, wherein the communication interface is configured to transmit data from the pressure sensor to a user device.

20. The RMT device of claim 17, further comprising an orifice defined in the vent, wherein the orifice functions as a bleed hole for the chamber.

21. The method of claim 8, wherein providing the visual indication of the results of the RMST and training exercises comprises providing for display of the results of the RMST and the training exercises via an application on a mobile device.
